(19) United States
(12) Patent Application Publication
(10) Pub. No.: US 2013/0123543 A1
(43) Pub. Date: May 16, 2013

(54) Polyacetylenic Compounds with Anti-Fungal Activity Derived from the Bacterium Collimonas and Methods for the Preparation and Identification Thereof

(71) Applicants: Kathryn Fritsche, Heteren (NL); Johannes Henricus Josephus Leveau, Heteren (NL); Wietse De Boer, Heteren (NL); Johannes Antonie Van Veen, Heteren (NL)

(72) Inventors: Kathryn Fritsche, Heteren (NL); Johannes Henricus Josephus Leveau, Heteren (NL); Wietse De Boer, Heteren (NL); Johannes Antonie Van Veen, Heteren (NL)

(73) Assignee: NETHERLANDS INSTITUTE OF ECOLOGY, Heteren (NL)

(21) Appl. No.: 13/640,304

(22) PCT Filed: Apr. 14, 2010
(86) PCT No.: PCT/EP10/54871

§ 371 Date: Jan. 22, 2013

(51) Int. Cl. C12N 15/11

(52) U.S. Cl. CPC C12N 15/11
USPC 568/300; 536/23.7; 435/132; 435/6.15; 435/6.11; 530/350

(57) ABSTRACT

The present invention relates to isolated nucleic acid sequences involved in, or capable of the biosynthesis of polyacetylenic compounds with anti-fungal activity and especially isolated nucleic acid sequences derived from Collimonas fungivorans. Further, the present invention relates to methods for identifying the present isolated nucleic acids, the use thereof for identification of homologous nucleic acids in other organisms or the use thereof for the biosynthesis of polyacetylenic compounds with anti-fungal activity. Further, the present invention relates to novel polyacetylenic compounds with anti-fungal activity. Specifically, the present invention relates to isolated nucleic acids encoding one or more proteins involved in the synthesis of a polyacetylenic compound with antifungal activity wherein said isolated nucleic acid comprises a nucleic acid sequence having at least 70%, preferably at least 80%, more preferably at least 90%, most preferably at least 95% identity with SEQ ID No. 1.

The present invention relates to isolated nucleic acid sequences involved in, or capable of, the biosynthesis of polyacetylenic compounds with anti-fungal activity and especially isolated nucleic acid sequences derived from Collimonas fungivorans. Further, the present invention relates to methods for identifying the present isolated nucleic acids, the use thereof for identification of homologous nucleic acids in other organisms or the use thereof for the biosynthesis of polyacetylenic compounds with anti-fungal activity. Further, the present invention relates to novel polyacetylenic compounds with anti-fungal activity.

The genus Collimonas belongs to the family Oxalobacteraceae, order Burkholderiales, class β-Proteobacteria. Presently, the genus Collimonas comprises three species designated Collimonas fungivorans, Collimonas arenae, and Collimonas pratensis. Further, isolates are described, presently designated as collimonads, potentially representing additional species belonging to the genus Collimonas.

First discovered between the roots of Marram grass, Ammophila arenaria, at coastal dune sites of the island of Terschelling, The Netherlands, Collimonas strains have since been detected in various locations around the world, mostly representing vegetated soils.

Collimonas bacteria are characterized by several commercially exploitable properties. These properties comprise, for example, anti-fungal activity, mycophagy, i.e. the ability to feed on living fungi, suppression of the fungal diseases tomato foot or root rot, chitinolysis, mineral weathering, efficient root colonization, and degradation of aromatic pollutants.

Of the above exploitable properties, of special interest are the anti-fungal activity and mycophagy observed for Collimonas species. The anti-fungal activity and mycophagy, i.e. the ability to grow at the expense of living fungal hyphae can be at least partly attributable to the property of Collimonas to be able to grow on fungi as a sole energy and nutrient source.

Inherently, the anti-fungal and mycophagy activities of Collimonas, and especially Collimonas fungivorans, can be exploited to treat fungus-based diseases of plants, animals and humans, undesired fungus-based decay, e.g. food spoilage, and undesired fungal presence, e.g. indoor fungi.

Project SCOLLIGEN (Sequencing the COLLImonas GENome) was initiated at the Netherlands Institute of Ecology to determine the complete nucleotide sequence of Collimonas fungivorans strain Ter331 and to discover the genes, enzymes, and regulatory networks that underlie the Collimonas phenome, i.e. the set of all phenotypes expressed by this organism with a particular focus on those phenotypes contributing to the anti-fungal and/or mycophagous behaviour of Collimonas.

The SCOLLIGEN project resulted in a fully annotated genome of Collimonas fungivorans Ter331 thereby allowing identifying genetic factors, such as genes or gene clusters, contributing to the bioproduction, or synthesis, of interesting biologically active molecules, and especially biologically active molecules involved in the anti-fungal and/or mycophagous phenotype of the bacterium.

Identifying suitable genetic factors, such as genes or gene clusters, involved in the biosynthesis of compounds involved in the mycophagous and/or anti-fungal phenotype, is further facilitated by the availability of two other resources besides the complete annotated genomic sequence. These sources are a Collimonas genomic DNA fosmid library and a Collimonas plasposon mutant library.

The fosmid library comprises Escherichia coli clones carrying approximately 37 kb fragments of the Collimonas fungivorans Ter331 genome thereby providing tools for identifying gain-of-function phenotypes. Because all fosmid library clones are end-sequenced and mapped to the genomic sequence, the nucleotide sequence and gene content of each individual member of the fosmid library is known or derivable.

The plasposon mutant library comprises randomly plasposon-mutagenized derivatives, or mutated clones, of Collimonas fungivorans Ter331. The plasposon mutant library allows for screening of lack-of-function phenotypes thereby allowing the identification of gene(s) or loci, such as gene clusters, involved in, or are essential for, for example, mycophagy and related properties of Collimonas such as anti-fungal activity.

The annotated genome of Collimonas fungivorans Ter331, the fosmid and plasposon mutant library in combination with a novel screening method allowed the identification, and isolation, of a unique Collimonas gene cluster, i.e. a genomic Collimonas fragment comprising several genes, of which at least one or more genes are involved in the biosynthesis of a unique low-molecular-weight compound designated as “collimomycin”.

Considering the threat of fungal diseases in plant, animals and humans, and the availability of only a limited number of compounds to treat fungal based diseases, there is a continuous need in the art for novel compounds with anti-microbial activity and especially anti-fungal activity. Accordingly, the present unique low-molecular-weight compound designated as “collimomycin” as well as the gene cluster involved in its biosynthesis are valuable means addressing this need.

Considering the above, it is an object of the present invention, amongst other objects, to provide novel compounds with anti-fungal activity and means for the preparation thereof.

This object, amongst other objects, is provided by the present invention by the provision of a Collimonas gene cluster involved in the biosynthesis of anti-fungal compounds, as well as the anti-fungal compounds themselves, as outlined in the appended claims.

Specifically, according to a first aspect of the present invention, this object is provided by an isolated nucleic acid encoding one or more proteins involved in the synthesis of a polyacetylenic compound with anti-fungal activity wherein said isolated nucleic comprises a nucleic acid sequence having at least 70%, preferably at least 80%, more preferably at least 90%, most preferably at least 95% identity with SEQ ID No. 1.

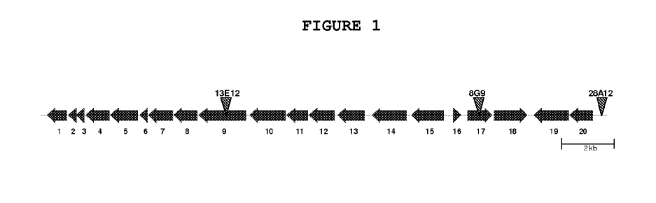

SEQ ID No. 1 represents a Collimonas gene cluster, or genomic fragment, of approximately 22.65 kb comprising 20 open reading frames (ORFS). A schematic representation of SEQ ID No. 1, including the open reading frames, is presented herein as FIG. 1. The function of several of the ORFS identified in the present isolated nucleic acid exhibit homology to known gene sequences of other species and, accordingly, at least part of their individual functions can be derived. Table 1, as presented herein below, summarizes the ORFS identified in SEQ ID No. 1 and their homology to known genes.

According to the present invention, the expression “ . . . one or more . . . ” comprises “ . . . 2 or more . . . ”, “ . . . 3 or more . . . ”, “ . . . 4 or more . . . ”, “ . . . 5 or more . . . ”, “ . . . 6 or more . . . ”, “ . . . 7 or more . . . ”, “ . . . 8 or more . . . ”, “ . . . 9 or more . . . ”, “ . . . 10 or more . . . ”, “ . . . 11 or more . . . ”, “ . . . 12 or more . . . ”, “ . . . 13 or more . . . ”, “ . . . 14 or more . . . ”, “ . . . 15 or more . . . ”, “ . . . 16 or more . . . ”, “ . . . 17 or more . . . ”, “ . . . 18 or more . . . ”, “ . . . 19 or more . . . ”, or “ . . . 20”

The term “sequence identity”, as used herein is defined as the number of identical nucleotides, or amino acids, over the full length of the indicated sequence divided by the number of nucleotides, or amino acids, of the full length of the indicated sequence multiplied by 100%. For example, a sequence with at least 80% identity with SEQ ID No. 1 comprises over the total length of 22.65 kb of SEQ ID No. 1 at least 18120 identical nucleotides, i.e., 18120/22650*100%=80%.

It is noted that the present invention does not relate to nucleic acid sequences having at least 70%, preferably at least 80%, more preferably at least 90%, most preferably at least 95% identity with SEQ ID No. 1 not encoding one or more proteins involved in the synthesis of a polyacetylenic compound with antifungal activity.

The above can, for example, be readily determined by transforming a Collimonas bacterium, or any other suitable host, with a sequence of interest and detecting the biosynthesis of the present anti-fungal compound, for example, by determining inhibition of fungal growth, such as Aspergillus niger growth, using a method as outlined in the examples below.

According to a preferred embodiment of this first aspect of the present invention, the present isolated nucleic acid comprises SEQ ID No. 1.

According to another preferred embodiment of the first aspect of the present invention, the present polyacetylenic compound with antifungal activity comprises an ene-triyne moiety according to the formula:

—C═C—C≡C—C≡C—C≡C— (1)

According to an especially preferred embodiment of the first aspect of the present invention, the present polyacetylenic compound with antifungal activity has the elemental formula C₁₆H₁₈O₄ preferably additionally comprising an ene-triyne moiety according to the formula:

—C═C—C≡C—C≡C—C≡C— (1)

According to yet another preferred embodiment of the first aspect of the present invention, the present one or more proteins are selected from the group consisting of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, and SEQ ID No. 21.

According to this preferred embodiment, the expression “ . . . one or more . . . ” comprises “ . . . 2 or more . . . ”, “ . . . 3 or more . . . ”, “ . . . 4 or more . . . ”, “ . . . 5 or more . . . ”, “ . . . 6 or more . . . ”, “ . . . 7 or more . . . ”, “ . . . 8 or more . . . ”, “ . . . 9 or more . . . ”, “ . . . 10 or more . . . ”, “ . . . 11 or more . . . ”, “ . . . 12 or more . . . ”, “ . . . 13 or more . . . ”, “ . . . 14 or more . . . ”, “ . . . 15 or more . . . ”, “ . . . 16 or more . . . ”, “ . . . 17 or more . . . ”, “ . . . 18 or more . . . ”, “ . . . 19 or more . . . ”, or “ . . . 20”

The present compounds having anti-fungal activity can be provided by expressing the present one or more proteins, as defined above, in a suitable host such as a bacterium, yeast or plant.

Accordingly, according to a second aspect, the present invention relates to a method for providing a polyacetylenic compound with antifungal activity comprising:
expressing one or more proteins encoded by an isolated nucleic acid according to the present invention and as defined above, in a suitable host; and
isolating said polyacetylenic compound.

Expression of the present one or more proteins in a host other than Collimonas, and especially Collimonas fungivorans, can readily be provided by placing the coding sequences of the present one or more proteins, as disclosed herein as SEQ ID NOs 2 to 21, under control of suitable expression control sequences such as promoter, enhancer, terminator and other transcription and translation regulation sequences, or in suitable expression vectors or expression systems.

According to this second aspect of the present invention, the expression “ . . . one or more . . . ” comprises “ . . . 2 or more . . . ”, “ . . . 3 or more . . . ”, “ . . . 4 or more . . . ”, “ . . . 5 or more . . . ”, “ . . . 6 or more . . . ”, “ . . . 7 or more . . . ”, “ . . . 8 or more . . . ”, “ . . . 9 or more . . . ”, “ . . . 10 or more . . . ”, “ . . . 11 or more . . . ”, “ . . . 12 or more . . . ”, “ . . . 13 or more . . . ”, “ . . . 14 or more . . . ”, “ . . . 15 or more . . . ”, “ . . . 16 or more . . . ”, “ . . . 17 or more . . . ”, “ . . . 18 or more . . . ”, “ . . . 19 or more . . . ”, or “ . . . 20”

According to a preferred embodiment of this second aspect of the present invention, the present polyacetylenic compound with anti-fungal activity comprises an ene-triyne moiety according to the formula:

—C═C—C≡C—C≡C—C≡C— (1)

and, preferably, the polyacetylenic compound with anti-fungal activity has the elemental formula C₁₆H₁₈O₄.

According to another preferred embodiment of this second aspect of the present invention, the present one or more proteins are selected from the group consisting of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, and SEQ ID No. 21.

The Collimonas gene cluster as described herein encodes one or more proteins capable of, or involved in, the biosynthesis of novel compounds with anti-fungal activity.

Accordingly, according to a third aspect, the present invention relates to polyacetylenic compounds with antifungal activity obtainable by methods as described above.

The present polyacetylenic compounds of this third aspect of the present invention preferably comprise an enetriyne moiety according to the formula:

—C═C—C≡C—C≡C—C≡C— (1)

and, preferably, have the elemental formula C₁₆H₁₈O₄.

The present Collimonas gene cluster as defined and disclosed herein provides for the identification of similar gene clusters or proteins in other organisms such as microorganisms, and especially bacteria, for example, and preferably, through commonly known molecular biology techniques such as hybridization, nucleic acid amplification, primer design and/or database homology searches.

Accordingly, according to a fourth aspect, the present invention relates to the use of an isolated nucleic acid, as defined above, or a fragment thereof, for the identification of nucleic acids encoding one or more proteins involved in the synthesis of a polyacetylenic compound with antifungal activity in an organism, wherein said organism is not Collimonas fungivorans.

The present gene cluster can readily be identified by using a novel high-throughput screening method based on the ability of Collimonas to inhibit the growth of fungi such as Aspergillus niger.

Accordingly, according to a fifth aspect, the present invention relates to methods for providing nucleic acids encoding one or more proteins with antifungal activity or one or more proteins involved in the synthesis of a compound with anti-fungal activity in a microorganism comprising:
providing an open bottom multiwell plate comprising wells with a suitable growth support substrate;
inoculating one side of said substrate with a fungus and the opposite side of said substrate with one or more genetically modified variants of said microorganism;
incubating said multiwell plate for a time period allowing growth of said fungus; and
identifying one or more genetically modified variants providing altered growth characteristics of said fungus as compared to the growth characteristics of said fungus in the presence of said microorganism not being genetically modified;
   wherein said providing comprises establishing the genetic modification in said identified genetically modified variants as compared to said microorganism not being genetically modified.

According to a preferred embodiment of this fifth aspect of the present invention, the present genetic modification comprises transposon mutagenesis.

According to another preferred embodiment of this fifth aspect of the present invention, the present microorganism is a bacterium, preferably Collimonas, more preferably Collimonas fungivorans.

The proteins as defined herein are suitable for the synthesis of the present compounds with anti-fungal activity.

Accordingly, according to a sixth aspect, the present invention relates to the use of one or more proteins selected from the group consisting of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, and SEQ ID No. 21 for the synthesis of a polyacetylenic compound with antifungal activity as defined above.

The present invention will be further detailed in the following examples of preferred embodiments. These examples are not intended to limit the scope of the present invention in any way which scope of protection is solely determined by the appended claims.

In the examples, reference is made to figures wherein:

FIG. 1: shows a schematic representation of Collimonas fungivorans Ter331 derived SEQ ID No. 1, showing the present gene cluster. The plasposon insertion sites of mutants with diminished antifungal activity towards Aspergillus niger are indicated;

### EXAMPLES

### Example 1

### Confrontation Assay Between the Bacterium Collimonas fungivorans and the Fungus Aspergillus niger

### Introduction

The genus Collimonas consists of soil bacteria that are known for their ability to grow at the expense of living fungal hyphae. Fungi that can be exploited by Collimonas bacteria experience a negative effect of the presence of collimonads on agar (in vitro antagonism). Collimonas fungivorans was confronted in vitro with the fungus Aspergillus niger.

During a first phase of confrontation, the Collimonas response consisted of the activation of several processes, among them the degradation of compounds of fungal origin, and the production of an antifungal compound; while during a second phase, it consisted of a generalized distress induced by the unfavourable environment created by the fungus. The response of Aspergillus niger was specific including a rearrangement of the lipid and cell wall metabolism and the activation of processes of cell defence.

### Materials and Methods

### Strain Cultivation

Collimonas fungivorans Ter331 was maintained at −80° C. in 1/10 TSB (1 g l⁻¹ KH₂PO₄, 5 g l⁻¹ NaCl, 3 g l⁻¹ Tryptone Soya Broth (Oxoid, Basingstoke, UK), 20 g l⁻¹ agar (J. T. Baker, Phillipsburg, N.J.)), supplemented with 20% glycerol. Aspergillus niger strain 400 (CBS 120.49) was maintained at −80° C. as spores suspended in 0.9% NaCl supplemented with 50% glycerol.

To prepare the fungus for the in vitro confrontation with Collimonas, deep-frozen spores of Aspergillus niger were plated on a 9 cm Petri dish containing PDA medium diluted with agar (19.5 g l⁻¹ Potato dextrose agar (Oxoid), 7.5 g l⁻¹ agar (J. T. Baker)) and incubated for 4 days at 30° C. Fresh spores were harvested from the fungal mycelium by washing with sterile saline solution supplemented with 0.005% Tween 80, filtered through Miracloth (Calbiochem, Nottingham, UK) to remove mycelial fragments, washed twice and resuspended in saline solution. To estimate the number of germinating spores, subsamples of this solution were plated on Water Yeast Agar medium (1 g KH₂PO₄ l⁻¹, 5 g NaCl l⁻¹, 0.1 g Bacto™ Yeast-Extract (Difco) l⁻¹, 20 g agar (J. T. Baker) l⁻¹).

In preparation for the confrontation assay, Collimonas fungivorans Ter331 was inoculated from 1/10 TSB agar plates into 1/10 liquid TSB medium at 25° C. to grow over-night. Bacterial cells were harvested by centrifugation, washed once with buffer solution (0.25 g l⁻¹ KH₂PO₄, pH 6.5), and re-suspended in buffer solution to obtain an optical density (OD) at 600 nm of 1, which corresponds to approximately 10⁹ cfu ml⁻¹.

### In Vitro Confrontation Assay

Petri dishes (9 cm diameter) containing 25 ml per plate of Water Yeast Agar medium supplemented with 10 μg bromocresol purple pH indicator per ml were used for the assay. In the center of the plate, an autoclaved 2-cm wide strip of Nuclepore Track-Etch polycarbonate membrane (Whatman, s'Hertogenbosch, The Netherlands, catalog number 113506, 0.2 μm pore size) was placed, on top of which eight 2.5 μl droplets each containing 10⁴ Aspergillus niger spores were equidistantly deposited. The polycarbonate membrane physically separated the mycelium from the agar medium preventing the fungus from growing into the agar and avoiding carryover of impurities into the biological samples.

Next, eight 2.5 μl droplets of bacterial suspension with an OD of 1 were placed at a distance of 2.5 cm on either side of the membrane and streaked into a single line parallel to the edge of the membrane. Plates were sealed with Parafilm and incubated at 20° C. Control plates were inoculated as described above, but with only Aspergillus niger or Collimonas fungivorans on the plate.

### Results and Discussion

The presence of Collimonas fungivorans bacteria on agar resulted in a clear inhibition of mycelium extension of Aspergillus niger. On the plates used for analysis Aspergillus niger was inoculated on polycarbonate filters. This inhibition was visible from day 6 on.

Growth inhibition was accompanied by browning of the mycelium. This phenomenon has also been observed during previously reported exposures of fungi to bacteria and could be due to the accumulation of melanin or alteration of fungal sporulation, two responses indicative of stress exposure.

Microscopic observations indicated that fungal hyphae in the presence of Collimonas were deformed and had increased branches as compared to the control. A similar phenotype was reported for the fungus Laccaria bicolor S238N when confronted with various bacterial strains, including Collimonas fungivorans.

During mycelial development, the medium became acidic, as could be seen by the change in pH indicator bromocresol purple in the area immediately surrounding the fungus.

Visible already on the third day, this acidic halo expanded gradually, keeping pace with fungal growth and covering the entire plate in about seven days. Acidification of the medium by the fungus was independent of the presence of bacteria.

After 5 days of co-inoculation, while the bacteria on the control plates remained visible as transparent biomass, the bacteria on the test plates became slimy. The production of slime coincided with the arrival of the fungal-induced acidification wave. During the course of the fungal-bacterial interaction there never was physical contact between the two organisms.

### Example 2

### Antifungal Activity of the Bacterium Collimonas fungivorans: Gene Cluster Involved in the Synthesis of Polyacetylenic Compound(s) with Antifungal Activity

Analysis of the antifungal activity of Collimonas fungivorans Ter331 revealed various degrees of inhibition against different fungi, Aspergillus niger, Cladosporium sp., Trichosporon guehoae, Fusarium culmorum, and Mucor rouxii, in agar plate confrontation assays (see example 1). A clear reduction of hyphal growth in the presence of Collimonas fungivorans Ter331 was observed, and microscopical analysis showed a remarkable deformation of the fungal hyphae.

It was confirmed that the anti-fungal activity was not affected by separation of the bacteria and the fungus by a dialysis membrane with a cut-off of 8-kDa. This indicates that the antifungal activity is not due to a protein or enzyme, but caused by a low molecular weight compound.

To identify the genetic basis of the inhibitory effects observed, a plasposon mutant library of Collimonas fungivorans Ter331 was screened for mutants with a reduced or diminished ability to inhibit the growth of Aspergillus niger.

For this, the agar plate confrontation assay of example 1 was adapted to be compatible with a 96-well screening format. This new assay comprised bottomless microtiter plates with in each well 150 μl of water-yeast agar containing 2 mM N-acetylglucosamine. One side of the agar in the well was inoculated with Aspergillus niger, the other with single plasposon mutants of Collimonas fungivorans Ter331.

Screening of approximately 3300 mutants of Collimonas fungivorans Ter331 revealed 7 confirmed mutants with altered antifungal activity towards Aspergillus niger. Two of these, 6B3 and 21E11 showed reduced anti-fungal activity, the other six, 10E11, 14C11, 14G4, 8G9, 13E12 and 28A12, showed completely abolished anti-fungal activity.

For the above mutants, plasposon insertion sites were determined. Analysis of mutants 8G9, 13E12 and 28A12, i.e., the plasposon mutants showing a completely abolished anti-fungal activity, revealed plasposons inserted within a DNA fragment of 15.5 kb on the Ter331 genome. Based on the plasposon insertion sites and the orientation of the predicted genes located on this DNA fragment a gene cluster was identified located on a 22.65 kb DNA fragment (SEQ ID No. 1) of the Collimonas fungivorans Ter331 genome responsible for the production of an antifungal compound.

20 ORFs were identified within SEQ ID No. 1 designated herein as SEQ ID Nos. 2 to 21. Comparison with the sequence databases revealed that homologues of 13 of the ORFs (SEQ ID Nos. 4 to 16) are present on the genomes of three Burkholderia strains: Burkholderia ambifaria IOP40-10, Burkholderia ambifaria Mex-5 and Burkholderia vietnamiensis G4. Seven of the genes (SEQ ID Nos 4 to 10) have also homologues in the genomes of Pseudomonas fluorescens Pf-5 and Streptomyces sp. Mg1 (Accession number DS570401). The clustering of the genes is similar to the clustering in Collimonas fungivorans Ter331.

While Burkholderia ambifaria IOP40-10 (Accession ABLC01000003) and Mex-5 (Accession NZ_ABLK01000019) are sequenced for comparative genomic studies of strains belonging to the Burkholderia cepacia complex, Burkholderia vietnamiensis G4 is well described as degrader of trichloroethylene and toxic aromatic compounds.

Streptomyces sp. Mg1 has been isolated from a glacier in Alaska but no further information is published to date. On the other hand, the properties of Pseudomonas fluorescens Pf-5 are very well described. The strain is known to suppress different fungal pathogens and the genes responsible for the production of several antibiotic metabolites, e.g. 2,4-diacetylphloroglucinol, pyoverdine and pyrrolnitrin, have been identified.

In the table 1, the genes of the present collimomycin locus identified are listed and the degree of homology of the derived protein sequences to published sequences (most similar proteins as well as the best experimental hits) is shown.

TABLE 1
Genes identified in SEQ ID No. 1 and homology of
their expression products

| | Most similar | | | |
|---|---|---|---|---|
| | protein | | Best | |
| | organism | | experimental | |
| | Accession Number | | hit | |
| | (gene ID genome | % | (organism) | |
| ORF | sequence) | identity | Accession No. | % identity |
| 1 | hypothetical | 53 | lactone- | 49 |
| | protein | | specific | |
| | Predicted | | esterase | |
| | hydrolases or | | Pseudomonas | |
| | acyltransferases | | fluorescens | |
| | Sorangium | | AAC36352 | |
| | cellulosum ‘So ce | | | |
| | 56’ | | | |
| | CAN93372 | | | |
| 2 | Hypothetical | | — | |
| | No hit | | | |
| 3 | Rubredoxin-type | 77 | Rubredoxin | 78 |
| | Fe (Cys) 4 protein | | Acinetobacter | |
| | Burkholderia | | sp. M-1 | |
| | vietnamiensis G4 | | BAB33291 | |
| | ABO56995 | | | |
| | (GENE ID: 4950229 | | | |
| | Bcep1808_4014) | | | |
| 4 | delta 12 desaturase | 65 | FMP | 42 |
| | Burkholderia | | Ralstonia | |
| | ambifaria IOP40-10 | | eutropha H16 | |
| | EDT06087 | | AAA21950 | |
| 5 | fatty acid | 82 | delta-12 | 54 |
| | desaturase | | desaturase | |
| | Burkholderia | | Synechococcus | |
| | ambifaria MEX-5 | | sp. PCC 6714 | |
| | EDT43257 | | BAA02921; | |
| 6 | phosphopantetheine- | 80 | polyketide | 65 |
| | binding | | synthase | |
| | Burkholderia | | (phosphopantetheine | |
| | ambifaria IOP40-10 | | attachment | |
| | EDT06085 | | site | |
| | | | Chondromyces | |
| | | | crocatus | |
| | | | CAJ46689 | |
| 7 | delta-9 acyl- | 86 | fatty acid | 58 |
| | phospholipid | | desaturase | |
| | desaturase | | JamB | |
| | Burkholderia | | Lyngbya | |
| | ambifaria IOP40-10 | | majuscule | |
| | EDT06084 | | AAS98775 | |
| 8 | Stearoyl-CoA 9- | 70 | fatty acid | 55 |
| | desaturase | | desaturase | |
| | Burkholderia | | JamB | |
| | ambifaria MEX-5 | | Lyngbya | |
| | EDT43254 | | majuscula | |
| | | | AAS98775 | |
| 9 | AMP-dependent | 67 | nonribosomal | 54 |
| | synthetase and | | peptide | |
| | ligase | | synthetase | |
| | ABO56989 | | Streptomyces | |
| | (GENE ID: 4950224 | | atroolivaceus | |
| | Bcep1808_4008) | | AAN85512 | |
| 10 | major facilitator | 90 | ChaT1 protein | 47 |
| | superfamily MFS_1 | | Streptomyces | |
| | Burkholderia | | chartreusis | |
| | ambifaria MEX-5 | | CAH10178 | |
| | EDT43252 | | | |
| 11 | hypothetical | 87 | — | |
| | protein | | | |
| | Burkholderia | | | |
| | ambifaria IOP40-10 | | | |
| | EDT06080 | | | |
| 12 | Rieske (2Fe—2S) | 95 | alpha-subunit | 49 |
| | domain protein | | oxygenase VanA | |
| | Burkholderia | | Streptomyces | |
| | ambifaria IOP40-10 | | sp. NL15-2K | |
| | EDT06079 | | BAF33363; | |
| 13 | fatty acid | 85 | carotene | 38 |
| | desaturase | | ketolase CrtW | |
| | Burkholderia | | Algoriphagus | |
| | ambifaria IOP40-10 | | sp. KK10202C | |
| | EDT06078 | | ABB88952 | |
| 14 | monooxygenase, FAD- | 88 | Halogenase | 40 |
| | binding | | Actinosynnema | |
| | Burkholderia | | pretiosum | |
| | ambifaria MEX-5 | | subsp. | |
| | EDT43248 | | auranticum | |
| | | | AAM54090 | |
| 15 | 3-oxoacyl-acyl | 92 | Pseudomonas | 78 |
| | carrier protein | | syringae pv. | |
| | synthase II | | phaseolicola | |
| | Burkholderia | | CAI36078; | |
| | ambifaria MEX-5 | | | |
| | EDT43247 | | | |
| 16 | similar to CG3523- | 61 | — | |
| | PA | | | |
| | Tribolium castaneum | | | |
| | XP_974066 | | | |
| 17 | transcriptional | 76 | Chain A, | 47 |
| | regulator, LysR | | Crystal | |
| | family | | Structure Of | |
| | Burkholderia | | The Effector | |
| | vietnamiensis G4 | | Binding Domain | |
| | ABO54218 | | Of A Catm | |
| | (GENE ID: 4952917 | | Variant, | |
| | Bcep1808_1207) | | Catm(V158m) | |
| | | | Acinetobacter | |
| | | | sp. 2H98_{—} | |
| 18 | major facilitator | 83 | MopB | 56 |
| | family transporter | | Burkholderia | |
| | Burkholderia | | cepacia | |
| | pseudomallei 668 | | AAB41509 | |
| | ZP_01764941 | | | |
| 19 | conserved | 66 | — | |
| | hypothetical | | | |
| | protein | | | |
| | Burkholderia | | | |
| | phymatum STM815 | | | |
| | ACC76384 | | | |
| 20 | fatty acid | 62 | RtxC | 43 |
| | desaturase | | Bradyrhizobium | |
| | Burkholderia | | elkanii | |
| | phymatum STM815 | | BAB55901; | |
| | ACC76383 | | Okazaki et | |
| | | | al., 2004 | |

Microarray experiments showed that the presence of Aspergillus niger in dual cultures with Collimonas fungivorans Ter331 on water yeast agar plates resulted in increased mRNA expression levels of 15 of the present 20 genes (orf2-orf16) present on the collimomycin locus as compared to cultures of Ter331 alone.

Collimonas fungivorans Ter331 proteins derived from the collimomycin gene cluster (SEQ ID No. 1) share 48 to 88% identical amino acids with homologs of Burkholderia ambifaria IOP40-10; Burkholderia ambifaria Mex-5 and Burkholderia vietnamiensis G4 and 41 to 81% identical amino acids with proteins derived from Streptomyces sp. Mg1 and Pseudomonas fluorescens Pf-5 genome sequences.

Some of these proteins appear to be conserved in Collimonas fungivorans Ter331 and the other strains, particularly the Burkholderia spp.; while the identity to proteins of other (micro)organisms published in the databases is considerably lower. That is especially the case for Ter331 orf's 5, 7 and 11-14.

These conserved regions allow primer development or design. These primers can subsequently be used to search for collimomycin-like genes/gene cluster in other bacteria and metagenomic libraries.

### Example 3

### Analysis of the Anti-Fungal Compounds(s)

The antifungal activity of Collimonas fungivorans was extracted with organic solvents from solid cultures on water-yeast agar with 2 mM N-acetylglucosamine. Anti-fungal activity was detected after extraction in bioassays with Aspergillus niger.

Collimonas fungivorans Ter331 produced anti-fungal activity on solid medium independently of the presence of the fungus as was shown by extraction experiments after cultivation of Collimonas fungivorans Ter331 alone or together with Aspergillus niger.

Extracts of agar cultures of the three mutants with plasposon insertions in the collimomycin gene cluster identified (i.e. 13E12, 8G9, 28A12) did not cause inhibition of Aspergillus niger.

HPLC analysis of extracts of cultures of the wild type Collimonas fungivorans Ter331 and the mutant 13E12 were compared. The extracts differed clearly in the peak pattern of the HPLC chromatograms. In contrast to the mutant, the wild type extract contained several compounds showing characteristic UV spectra with multiple maxima. The absence of that group of HPLC signals in the mutant demonstrated that one or more of these compounds are responsible for the inhibition of Aspergillus niger by Collimonas fungivorans Ter331.

The UV-spectra observed are characteristic for polyacetylenic compounds and they allow the prediction of the pattern of C—C double and triple bonds in such compounds. Based on the maxima of the obtained UV spectra, the crude extract contained compounds with ene-diyne, ene-triyne and diene-triyne groups.

HPLC fractionation of agar extracts followed by bioassays with Aspergillus niger showed that there is more than one active compounds present in the agar extract of Collimonas fungivorans Ter331.

Results indicate that two to seven related active polyacetylenic compounds produced by Collimonas fungivorans Ter331 contain an ene-triyne group and elemental analysis of these active polyacetylenic compounds indicated at least one compound with the elemental formula C₁₆H₁₈O₄.

1. An isolated nucleic acid encoding one or more proteins involved in the synthesis of a polyacetylenic compound with antifungal activity wherein said isolated nucleic acid comprises a nucleic acid sequence having at least 70%, preferably at least 80%, more preferably at least 90%, most preferably at least 95% sequence identity with SEQ ID No. 1.

2. The isolated nucleic acid according to claim 1, wherein said nucleic acid comprises SEQ ID No. 1.

3. The isolated nucleic acid according to claim 1, wherein said polyacetylenic compound with antifungal activity comprises an ene-triyne moiety according to the formula:
—C═C—C≡C—C≡C—C≡C— (1)

4. The isolated nucleic acid according to claim 1, wherein said polyacetylenic compound with antifungal activity has the elemental formula C₁₆H₁₈O₄.

5. The isolated nucleic acid sequence according to claim 1, wherein said one or more proteins are selected from the group consisting of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, and SEQ ID No. 21.

6. A method for providing a polyacetylenic compound with antifungal activity comprising:
expressing one or more proteins encoded by an isolated nucleic acid according to claim 1 in a suitable host; and
isolating said polyacetylenic compound.

7. The method according to claim 6, wherein said polyacetylenic compound with antifungal activity comprises an ene-triyne moiety according to the formula:
—C═C—C≡C—C≡C—C≡C— (1)

8. The method according to claim 6, wherein said polyacetylenic compound with antifungal activity has the elemental formula C₁₆H₁₈O₄.

9. The method according to claim 6, wherein said one or more proteins are selected from the group consisting of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, and SEQ ID No. 21.

10. A polyacetylenic compound with antifungal activity obtainable by a method according to claim 6.

11. The polyacetylenic compound according to claim 10, wherein said polyacetylenic compound with antifungal activity comprises an ene-triyne moiety according to the formula:
—C═C—C≡C—C≡C—C≡C— (1)

12. The polyacetylenic compound according to claim 8, wherein said polyacetylenic compound with antifungal activity has the elemental formula C₁₆H₁₈O₄.

13. Use of an isolated nucleic acid according to claim 1, or a fragment thereof, for the identification of a nucleic acid encoding one or more proteins involved in the synthesis of a polyacetylenic compound with antifungal activity in an organism, wherein said organism is not Collimonas fungivorans.

14. Use according to claim 13, wherein said identification comprises one or more methods selected from the group consisting of hybridization, nucleic acid amplification, primer design and database homology searches.

15. A method for providing nucleic acids encoding one or more proteins with antifungal activity or one or more proteins involved in the synthesis of a compound with antifungal activity in a microorganism:
providing an open bottom multiwell plate comprising wells with a suitable growth support substrate;
inoculating one side of said substrate with a fungus and the opposite side of said substrate with one or more genetically modified variants of said microorganism;
incubating said multiwell plate for a time period allowing growth of said fungus; and
identifying one or more genetically modified variants providing altered growth characteristics of said fungus as compared to the growth characteristics of said fungus in the presence of said microorganism not being genetically modified;
wherein said providing comprises establishing the genetic modification in said identified genetically modified variants.

16. The method according to claim 15, wherein said genetic modification comprises transposon mutagenesis.

17. Use of one or more proteins selected from the group consisting of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, and SEQ ID No. 21 for the synthesis of a polyacetylenic compound with antifungal activity.


SEQUENCE LISTING (21 sequences)

SEQ ID NO: 1 - DNA, 22650 bp, Collimonas fungivorans
agaatgtctcatgctgccggaggttgctgtcgcgatgatcgggcatgttgcgctggctgcatagccatatacggccctcctcaggccaccgcggtgcgcgccatcaggaaagagcgcgcggcttcgattacttcaggcgcctgcaggatgcggcgatggcccaggccgtcaaggcggagcagttgggcaccttgccaggcattggcgatttcgaccccgagcgataccggcaccacgcggtcgtcgttcgaatgcaggatcagcaccggctgcttgagattggcagccgccttcggtgtgtcgatatcggcgacatcgacgccttgctcttgcagcaaggcgatcatggccgctacctggtggcggtcgaggccggcctggacgccgaagccgacggcataatcgaaacagcgcgccggcggtgcgatcagcaccaagcgctcgacctgcaggccgtgccgcactgcttccacagtaagagcacagccgaccgagtgggcgattgcggcatacaccggaccgatcaggtcctggagtttcagcagcgcggcggcgcaggccgggattgacgaagtcgttccttccgactcgccatgcgccggcaggtcgactagcaccacgcggtagccggctgccagcagcggtggtgtgaacgacgccatgtcgctggagcggccttcccagccgtgaaccagcaatactatcgggccacggccggcttgccaggacaccagcttgccggtggggccgttggtttccacccggctcgcgccttggaaaccggcagtgtccggggcacgctcataagttcgcggcgtgaggaacgccttggcaacggattcgggggtaggcgctaactgttcggcgggggtgctcacgatgatggtctccttttgatattgtggtttgcggtctggtcctgctgcgtgcaaccggcgccgttacgccgatttcacaaggatgggccatatgcctgcgtttcctttttttgcagcagcacatcgaggatctggcgcccggcaatcgaggcctggcccactgccaggtggatgaacccggcgtcgttatccaagttttcctaggcctgcggcagatatagatgtcttccgcgatattgatgcgcaggtccttggcgatggcgtccgaaagctgctggttgtcgatgccaaggcggtagtcggtatttgacatgggcgtagaacatttcgccggcgtagcgcgtgcttgttcaggacggttcttgggaacagttcggccttgatgtcggaccggcctgggccgccaggaaatcttactgccgccgccggttttcaacaccctgtcaaaattgataggtttgcgtgcgactgtggcaactctgctgctgcaactgcctagaattccaccatggtgaaatcctctttcttgacgccgcactcggggcaggtccaggtgtccgggatatcgtcccagcgggtgccggccgccaggccttcgctgggcgcgccgtccttttccgagtagatccagccgcacaccagacacatcagtgtcttgaattcagtttctgtttcagtcattgctaggttccaggctagttggggtcagatgggatgggccgatgaagcgggtgatcagttccttgaccggtgtggcagacagtatccggctgtggccgaggccggaggtctgctccaggcgtacgtttttcaggctgcgcatcagcctttgggcttcgctgagcggcaccaccggatcgtgctcgtcgtgcaccaccagcaccggcacctccatggccgcgcccagcacgctgatatcccagtggctgacgggaacgccgttctgcacctgcaggcgctcgtagatcttgccgatcacggccggcggcagctgctgatgctggttggtcgaccagcgctccagcacggtggacagggataccggagcggctatcagcaccaggcgtttgacggcttccgcgtgccgcgcgtcggcatgggcgatggccgctacggaggcaatcgaaccgagcgaatggccgacgatcacctgcacgttgtccagcgacttgatggcggcaccgaccgctttgacaaagcgcgtcatggtggtcttgttgccggccgattcgccatgcgcgggcgcatcgaacgaagccacctggaaaccgagcgccagcagcggcttgacgaaaccgatcatgctgctgctgtccgagccccagccgtgcaccagcagcacggtaggaccgtggtttttccagagataaccgtggcgcacgtcgtcgttgccggcgatctcgaaagtctgcgcgccgagcggcatgcggaacaaggtacggccgcctttcgaacgcgagcggccgaaggcgtccgccgcaatccgtccggccagggctggcgccagcatgccgcccagctggtaacccgccttggccaggctggcgccgatgccgagggccggcgccgccgccggctgttgcggctgttgtgacagcggcggcgagggtgaagtgatgtcagtcatgtcgtcaggtcctgcggttggcccggtacgggttgttggaggatcaggtattggcgacggaattggcgccgccgttaacctcggcgcgctgttccgccgaatccttgtaataaccggaatcgagatcgtagaaatggcagttcctgatgacccatagcaggtcgccgaagctgaaatccttttccctgatcatgcccggataagcctgtttcagcgcgacctgcgctttctgcaggttatagaatggcaccaccggcgccacgtgatgggcggtatggatggagatgttgtgggtcaggaacagcagccatttcggatagcggtaatcggtggtcaccagcatgcggctggcgttcggcgtccagtgttcggacgtcaggaacgggatatcggccgaggtgtggtgcatcagcgtggtgacgctgaaccaggtatgggtcgcaatccacggcgcaaagaacagcaggaagaaacctgtgatgccggtaaagtagatcagcgctgaaaaatacacgatcgaagccagcaccacgaaggcgatcgaacgcttgacgtcagtgcgcatatccttcttgggaaagaatcccggccggaagcccgacacgatccagtagttgatggtgccggcccagaacagccaggtcctggtgcccatgtagacgaaacgctggccgaacgacatgcgcttgtacatgtcgcgcgggatcggccgccagtcggtatcgagttcaaggttgttggtgtggaagtgatgcagattgtgcacgtgcttccatgaatagaacggatacagcagcggcagcagcgacaggtggccgatgacgatgttgagcttcttgttgcgcgagaacgaattgtgcccgcaatcgtgcgcgatgcaatgcaagccccaaccgcccaggccggcgatgatgtacaggggaatatagaatccccagtgcggcgcataggcgatgccgacgatggcgccgatatacagcgcgtagctgatgaaaaagccggccaggccgcgccaggcctgcggttcgaaatacgatttgggaatcgcttcggccaggcgggcgccttccagcgtcttgctcttgtccaggaacgcctcgaactcgggccccggcgatgaaggataactatttacggacggcattgatttctcccagattatgcgatggcgttgagcccgagttccgtggccaggtgagcactcagttcgtcgatgctcgggtgttcgaacagcagcgccggcgacaggcgctgctcgaccagtttttccaggtcgccggaaacccgcaccgcgacgatcgaatcgaggccgtacagttcgaacttcttggcagtgtcgatggcgctcgggtcgaccttgacttgcttggccaggcgcttgaccagccactggcggatcacttcctcgcttaacggaacattgctttcaacttcctctccgcctttgcggaagaagccgaacaggccgcccgatttgttttcttgagacatggtttctcctgggatgaatgtgaactaagaaatcttgctggatttagcgggcgcgcatcttggcgacggctttttcgtcaggcgagcgcaggttccagaccacgccgatcttgccgaaaccggccagcagccagccgctcaggtcgggctcccaccatttgacgccgtggcggtacgagcctgggaacgcatggtggttattctgcaagccttcgccgaaagtgaggatggcgacagtccagttgttggcgctgttatcgtcggtcttgaacggtttgccgccaatcatgtggcacaccgaaccgacgcaccaggcagcctggttggccaggaaaatccgcgccagcccgccgaacagaaaaccgctgaaagccgcgtcccagctgccgccgaccgcgccgccgatggcagctggcagggccagaccgagcacgatccacagaccgtaggtgcggtggtaaaagaacagcttgcggttggcgaggatgtccggcgcaaagaaattccagccggagacgtccttggccagcatccacggcatgtgcgcataccagatgccgcgcaggcggcccagcaggcccggtccgtgcaggttgggcgaatgcggatcgccgtgctggtcgctgtaggtgtggtggcggcgatgggtggtgacccagaacatgatcggcccttgcgccgccatcgaaccgcagatcagcagcaagccctcgaagaacggcgaggtgttgaaggtcttgtgcgccaggtagcgatggaaacccatcgtgatgccgcccatgtggaggaaatagaaaacgccgaacagcaccaggtcggtggtcgacatgcgtccttccagcgcacgctgcagcgccagcgcgaatcccgccagcggcaccagcatgaccaccagcgcggtaatctgcttgatggtggcgccggcgccggtcagcggtgcgacgcccggcctcagtgcaggaccggcggcgctcgccggtttgggaggtaagacattggtttgcgtagtcatggcgtttccccgacgttaatcgttattgaaaagtttctggtccagcggatcggcgctctggcgttggcggatatcccaggccaggccgagcagtcctaacagccggatcacccaggcggtggtgtcgatctgccagaaatggaaatcgttgtgcgccagcgccgggttggcgtgatggttgttgtgccagccgccgccggccgacaccagcgccagccagccgatgttgccgctggtgtcgcgcgtctggttgggccgcttgccgaaggtatggccgatcgaattgaccgcccaggtcacctggtcgagcaggaagatccgggccaggccgccccacagcagcccgccgactgcgccttcgacgccgctcagtgcggcgccggctgcggccggcaacgccaggccgagaaacacccagatcaggtaatgctggttgacgaacagcaccagcttgctgccgaacaggtccggcacataggtgctccagttgctgcgcttgacggtaaacagccagccgacatggccgtgccacaaggctttgatgcggccgcgcaggccaggcgacagcacccgcggcgaatgcggatcgccgtcctggtcggtgaagacgtggtgcatgcggtgcgtcgccgcccagaacaggatgggcccttgcgccgccatgcagccgaggatgccgataaccgcggtcacgaccggcccggccttgaacgcgcggtgcgaaaagaagcggtgcaaaccgccctcgacgccgaacgacgtcaggaagtacatgacgccgaacaggatcatgtcttgctgcttgaagccgaactggatcgaataccacagcgcggcgacgaaaccgacggcaggcgtcagcacggtcaggtaagccagccggcgagccttgctatcgccatccctgagcgcttgcactttctcgccaggcgctgcggcagcgggatttttggtatcgatcactgcttacccctgttggaaaagtgtggtgctgtcggtttccgtcgtcgttgcggatggcgtcggacgggccttggcgtacgccagcgagccggcgttgaaggcatcgcgacaggcgctgcggcgcaccttgccgctggttgtcagcggaatggtcgacaccggcgccaggtgcacggtgtgcggcgccacgccatggctgcgggtaatcgcggcggcgatcgcgtcttcgacgttttccaggtcttcgccgctcagcttggctgagcgcaggatctcggccaccaccaccagctgttccgtgtcgtctcgtgtcaccgaaaaggctgcgacgccgttggtgcggatcgccgggtggctggcctgggctgcggcttcgatatcttgcggatagaggttgcggccggcgaaaatgatgacgtccttgatgcggccggtgatatacagttcgtcgtcgagcatgaaaccgaggtcgccggtgcgcaagtaggggccgtcctgccgatcgtcggcaatgaaggcatggaatgtttcggtgctggcttcgggctggttgagataggcttgcgccacgctggcgcccttgaaccacagttcgccgaccgcgccggccggcagcggctgccgggtgtcgggatcaacgatggccatcacatgtccgctggctaccgtgccgcagctgatgaccgccgtggcaggcgcatctgcggtagccggcttggcctggccgcaggccagcgcttcatggtccagcagcagcgtcttgggtgtggcgtcgcggtttgctttgccggaaatgaacagggtggcttcggccaggccgtagcacgggacaaacgccgcgctggagaatccggccgctttgaatttttccgaaaaacggtcaagcgtggcttgacgcaccggttcggcgccgcaaaacagcatcttcagccggctcaggtcgagttcggcaatttcttcatcggtaatggtgtcgacgcacaggtcgagcgcgaagttgggcgctaccgaggtggtgacctggtgcgtcgacaagcctttcagccagcgtagcggacgctgcacgaaatgggcgggcgtcatgaccaccagcgtgaatccgctgtatacggacagcagcagtgctcccatcaggcccatgtcgtggtagggcggcagccaggtgaagccgatatgtttttctgccgatcccagccggatgtcgagcacgcggctgttgctgatcaggttggcggcggagagcaccacgcccttgggattgccggtggagccggaggtgtattgcagcagcgccgggaactgttcactgctgcccgcttcatcgttctcatcagtgggcagcggttgggtgtcctgcgcttcgaaaaaatcgctgccgacaaacagccattcaggaacattgccctcggcggagagcgaggcattgaggcggtccacggctgcctgtagatttccttcggcgatcaccagctgcgcattgcagtcattgcagatgctggcgaagcgcgccaccgcgcggctgccgaccggcgggaacgacggcacggcggtcgcgccggccttgaaaatggcgaacagcgcggtgacgtagtgcaggcctggttccagcaccagcagcacgcgcgctcccggtgtcagttgctgtttgagctggcgcgccaggccggcggcgcgcaagtcgagctggcgataggtcaattcggcgatgtcgtcttcgccgtttttcaggaagcgcagagcaacctcgtccggcgtcgccgcggcgcgccggcgcaggatcgcatccatgtcgatatcctgcgcggcctgttccgggaccaatactgtcgcttcgctcatgctcaagctggatgtcattcgatactccgttatttgattgccgggaaaacatcacgccgccgagtaaagtccggggttgcgcttgtttggctaattttttatcactatagaaatatcaatacttcccaaaacactatttacagacatgcgctaagcgacggcgcgccagattaatgcgccgtcgcaggcgcaggtgcccgggatcgcgtgtcgactggtacgcacaggattgcggtggcaaaaccaagcgcgatctgcaggtaacagagggtgtgaaatgcggcgaggttcagcgtcgcatggccagttatggcgaccgtgagcgccaccccgagcaccgatcccatctggcgtaccgcctggttgacggcagagccaacgccgaaccgggcaggcggtaaatgcgcgacggcggccgccgacagtgaaggcatgaccatgcctacgcctagtccggtgagtagcgcccccggcagccaggtgtgcagatagtcgggattgatcccgggtaccagtgcgaaccaaatgctcgccgccgtggatatcaggcttccggtcaccagcagtaacttgtgccccgcacgcgccgcgaaccgtccgcacacgatcgccgtggggacgacgagcaacggcccaaggctgccagccagccccgcgcgcgtcagcgaatacgaccagacacctgtggtgaataggaacgtctggaagaacatcatggcgaaaccgatcgcgaagaacagcgatgcaagattgatatagcagtacgtccggtcctggaacagcgagagatcgatggcaggggcacgcacggtgcgcgcccagacgacgaacgctcccagcgtcgccagcccgcccgcgattgccaggatcaccatcggggacatccagccgaccgcctcggactgtaccaggccgaaagtgatcgcaccaacgccgaggatcagcaggacgacaccgatgacgtcgatcggcgcgccgcgctcaggatcgcgcgactcgtcgagaatgcgccacccaagccatagcgtgatcgcgccaagcggcaggttgagaaagaaggcccacggccagccgaagcgatcgaccaggaaagagccgaggctcggccccagcgcacctgcgaccccgctgactgcgccccacaagcccacggcgaccgcgcgtttgctggcgggaaatgctgccagcaagatcgacagcgatgccgggaacaatagcgccgcgccggctccctgtgctacgcgcattgcgatcagagtcgcgacgtccctagccaaaccgcagccgagggatccccccaggaacatggcaagtcctagcaagaacgtgcgcttgcgtccgcgcaggtcggcgaagcggcccgccggcacaagcaatgcggcaaagaccaacgtgtacccggtcaggacccacgacaggtgcgccgagccggcctcgggaaacgcccgttgcaaagcgggaaatgccgcgtagagcagggtgacgtcgatcgagacgaggaagaccgccacactcgcgatccaaaaaaccggccacggcgaaatcgagctcgactgcgcctgcagcgccggggctaggggaacgttcattacttgtacctcatcatgctggaggggaggtagcgccgtcgcccgactcgaagtacgaggcatcgagaaacgagagcgttcgcgcttgcatgcgcgcttgaaacagtgcgacgacgggccaagggcgcatcgccattgccatgaccttcacatgctgcgcatcctcccagtggatcacgttcgccacttccagcggcagtccggcgaccgaccccgtccacaggctcagcgtcgtggcgcctgccgtcaggcgcggcccgtttgcgcgtcctccatagacgacgattgagtgaccgcacagtcgttcgacgtaggcggcaccacgaatcggcttgcaggctacgggactggtcaggaacgcgttggcagctagaccgaacggcaacttggcgtccagcacttcgtcggtggggatcggcggcaacgcggcatcggttccagggggaagcaaccatgcgtcggcgtcgatgagatcccggcacgcgtcgtgcagcgcctgccgaaccggcgcggggaacgaggcgaggaagatgtcgacctgtacgcgctggccgaaccgccgtgccaccgtcaccccacgaatgcggtagtcctggaactgtccatgccactcgcgcccgccaatatcatcgccgctccatgtgtgatccggctcgacctcggaaaatatgctctcgacggcagccgcgagctgttccacccggccgtggccgacgacttctccgaacgtcatgggatgccgcaaactgacctcgtcctgcgccgacgtctcgagctcgcgcaacaactcatcaacatagagaccagcgtgtctttggcgactcatcggcgtttctttctctgtgagggtagcgttcatctatttatacctcttcaaactggggcaggggcgtcccgtggcccgttcgtatggaggcgtttgcgcccattcgctccagtatccgccgcgccgcgaacgctggccgatcaaggtggatatcgatcatcggtgcagtgggactccagttcgcctgaaggctttcggcgacgtctttgtcctccatgacggttgagaacacttctttcgctgtgaactccgtaaacccgtggttgtcgatatcgaaatcgcgatgcatgcccacgaagacgtggctcgtggtgtcggtggcgggagtgaagatcgtcgtcgtatagacgtgaaagaccttaccgtcggcgtcgtcgcacgcgtgggctctcgtttcgacccgcgtgttgccgacaggccagtagaggacctcctggaagcggtcgatgtagtcgagttgcaggatccggctgtacagcggaggcgttttctcgcggcgcatgtagcggcgcacgcggacttcgtcgtcgttaatcacgatttcaggcggagtctcgacgatgtcaagtgagcctagtgttttcgcgtgaacgaaggcggcgtgcgagatgtcgaggatgttgtcgattccgaacacgtagttgcaggcgatggtacagtagctcatcgtccccgcgagctcgggcgccgaacagacccaatggttcgggatcgcgctctcgtctgcctggtccgggtcgccgggccacagccagacgaagccatgcttctcgacggcggggaacgaacggacgcgcgcgcccttcgggatcgtgctctggctgggaatattgacgcactcccccgaacagtcgtagcggagcccatggtaccagcactgaagttcgtctccgatcaggcgtccccgcgacaacggaacttgtcgatgggcgcagcggtcctcaagcgcgacgacttggttcgctgaggtgcggtacatgacaatcttctcattcaggatggttcgcgcaaaaggctcgctcttgatttctccagaaagggctgccgcgtaccacgcattttggataaacatgatttttccttacttcgccgcgtacacggcactctggttccgggatagccgacccgaggtcgggatgatatgggcgttcaagatggtcctctgcttctactcgcgcacgactgcgaccatcgcttcgccggctcgcgtacccgccgagtctgcttcgcgatcgaagcgaccgtcggcaatccgttcgaggaaatcgtccgccttagacggtaattcgacgtgcgggtaagggaagcgccctgtcgtatagcgcagcgcgccaaaaaacgaccgctcaacctgagcgcccgtcgctttgatcagtccttgcgacgctaggtgcccgtgcaatgcgggcagccggtaacacgggacggccggatacaggtggtgctcgagatggaagttgcttccgaagaaaagcgctgtgtagatgggtgatgtgtagctgcgggcatcgtggtatcgacccggcgcggtgccggtgtgttccaggtacgcgcgcatggcgctcatcacgtacaacccgaaatacgggaccagcattaccgtgaaaccaagccacagcgagcgatgccagatgagcgcgtacaccgtcacgccgcataggccgagggcgatgttgatgcgggtgatccagcgcatttcgcgctcggagaacggaagctttgtgttctccggccaaggcaggcccagtgccatgcgtaaggcattcttcgtgtaaacccggacggtcccggaccgcgcgagaaagaagcgtgacacgaagttgttgtactgcgagtaaatctgtgcatcggggtcgatgtcctggtcggtgaagcggtggtgcttccagtgggtcatgctgaagcccacgatgaagaagaacggcaccagcgacgaaaagagtaccgccagcgccgcactgatgtacttgtttcggtgcaggttcgtgtgcattccctcgtgtccaacaaaggtcaagaggtgcattccatggccaccgatgattcccaggacgagtgccaaacctatccgcaccggcgtcgaggctggaatatcgacgatggcgaaggccaccatggccggaaccaggaagaatgcgagggcgtgcgctagatagagtgcggtggcgaagtgactggttcgatgaaatttttctggaagcttgagccgcgcaaacgcggtgtcggcctccaacgatgcttcgatggtcatagcctatctcctcgggtgacttaacgtaggtcctgtgaatgctggctgggcatcaccaggattggaaattgcgtggcctccaggccaatcggcgcggcatgccgagaccgatctctgccgcagcctcgaccggaacgtcgtcggtcatcattgagaaattgcatgccggatctcgggcgtgcaatttccgccaagcctgctgcgatgacttccgcgattgtccggtacgagtcggcgccgacgttatttgtggtcataccggctcgcttacgccgagggcgtggtcggcgctggatgcgagcaggttcgattggtcgaactcgggcgcccggactggggtggcgttagcctgctcggccaggcgctggcgtaacgccatttcgctgcggatctcctcgcctctccgggacatctcgatcaaaggctggatcacgcccacctcgccacgctcaacggcctcatctgcccacttcttaatgtcgctggggttgaatacggcaaacggcgagcactggcgattctgggcggagagcaagcgcgcctgcagcgtcgggtccgccgccagcttgccgaacagcatccggctgagcgggttgttgttggtgagcgcgccgcggctgcgggagtattcgaagaggtcgattgaagtcgcgtctcgctggcgccaatagcgtaccagcgcctggtccccgccctcgatgatcgcccgccccagtgaccgggcgtcgcgaagcgcgtcggtgatcccgaagccgggggacggatccatgaagaggcctgcgtcgccaaccagggcccagcccggccccgccgcctggcggaagaaaaagcgcaggttcaatgcgccgagcaccttgctcttaggctctccagcggtcagcggcgccgtgtaagggttgcgctggagcgtctcctgcagcttctgtagtgctcgccccttccactccggtagctgcgttctcgggaacgccaccccgatgagcaagagatcgcggttggttggaaagatgaagacgacgtcgtcgccgaagctgcagttcatcgccccccccaggtggcgcggatcgtcgtcgaaccaggtcggtctcggccagtacgcccagtacatcgcgcgtgggcagtcgtacccgtgatactcctcggcgctgacgagttgcgctgtggtcgagtgctggccatccgcgccgaccacgatccgtgcccggatctcttcgcgcgtcccggcgtgttcgaccacggctccggtgacccgttcgccgtcgcggagcagctcgaccagcttggtacgaggacggatctctgccccggcggcccgcgcttcgtctgccaggagcgcatcaagatccatacgacgcgggcaggagcagcgcccgccggccggatacacaacgcggccgatctcctcatcggcgccgtacatcatgctcggcaccggcggtgcgaagcgacgcaccttatcgccgaggcctaactcgtccagcagagccatgccgtggggcgaaatccagtgggtggacagcggttggtcgctcggcagattgttcgcatcgatagcgaggacgctcttgccggcgcgcgccaggtgaatcgcgagtgtggagcctgcgcagcgggcgccaacgattagggtatctaacacggagagattccttttatagaatgactagaactcggattgcgcgcgacgcggcctcgccatcccgcgcaagcaatgtagatgggcgcatcgaccgccggccgcagactggttgtcggcagccgggcattttcgttttcggcatgaacggcgtaatcaacgcacattctccttccgttcagcggagaaccgttgaaagagcaccgaggcgttgactccgccgaacccgaacccgttggacagcgctatctcaattggggcgcgacgcgccgacggccctatcagatcgaggccaatcgcggaatcgtcgagatgctccagattgagggttccgggaagcaccccatccctgagagcaagaactgaaaacgctgcctcgatggcgcccgccgcccccagcaaatgtcccgtcgcagacttggtagaggaaatagccggccccgtgctggttccgaagatagtcttcaaggccgcaatttctcccgcatcgccaaccggcgtcgatgtagcatgggcgttgacgtagtcgacacgctcgggggctattcccgccatcgccagggccagcctcatcgcccgcatggcgccattcccatcgtcggggccggccgtcatgtgataggcatctgcggtggttccatagccaatgatttcggcgatcgggatggcgccgcgtgctatggcatggtccagcgcctctaccactaacattgccgcgccttcacccatcacaaacccgtcacggtcgcggtcgaatgggcgcgaggcgcgaacaggctgttcgttgaagcccgtggacaaagcccgcgccgccgagaatccgccaaggctgactttgtcgaaagctgcttcggcgccgcctgccagcacaatatccgcttctcccgcacgaattaggcgcatcgcatcaccgatcgcctgtacgctagccgcgcaggcggtcacagggcaaccgagcggcccccgaaaccgatgcttaatcgatacttggccggccgccagattcgccaggaaggacggaatggtgaaaggcgacagccgccgcacgccaggcgattcacctatccttaccgcattggccagtccgagaaaaccgccgatgcccgacgcaatgatggtcgcagtgcgctcgcattgatggtcggtttgcggagcccatccggcatctgctagcgcctcctcagcggcgaccattgccatctggatgaagcggtccatctttttcaactctttgctcggaatggagatttcgggtgcgaaaccgccctcgggatcggctgtcctgtcttgcacggtaccgccgaccttaacaggaagatcgccgacaatttcgtctcccagcacgcgaagtccggaacgtccgtcaattaggcgctgccataccagttccgtgccacaacccagtggagaaacaatgcccagccccgtcacaaccacacggcgcatcattctatctccctctctcgcagcagggcccgccgagaggcatcggtcaagcttttcatcttgcgacgcagctctttagcttccgctttccccagcttcagaagaaattgaacatcgttatagttaaattccgcagtcatttcaccatccatatacatctgggtaatcattcgcaactgtgacgccgcgaccaggaaccacatccagcaagacatccgaaaactctgtcgcattaaggtttttataaaaaattcatcatttaaattcactgatctgtgaagccaaaaaatccttaatgtgacaaaacctaaattcgccgcacaaaatagataggcaattctgtaaattttatgtagcgtgttgccactcttctgaactgctttagttgggtgaaaaaacgttcaatcaaattacgattgcgatactggcgccggtcaaattctcgctgttctttccgattctttctctgcgggataatggccgcggcattttttattgcaatacaagcaagctatttgcaacatcaaaaatttgccttgcaaaatcgggatggctatgaattttttggcagcgccagcaataagctggtgagctcgcagaatcgaactatcgagagacacttcctcaaaacaacatcttcgggtgccccgcaaaagcgttctgccataccttcatgtccggccaccaacaccattccgtgggaaaattcactggacaaatcgcgccgcggactgtccgtgctatctgcggcaccatctcaacaaatgacctgcccgacatatcgcatactgtccgagccgaaaatttgaaaacaacagaaccctcttggaacgcttgaattctcgcctgaattcctataagtttccaataatcaattattatcaggtatataattccagctaatgatgaacatggccgacccaataaaaccaagagtcaaatcgagcttgcttaacgatcgcctcgactggaatctgctacggacattcatggtgattgccaaagagcgcagcatcagtcgggcggcaggtaagctgcacctcacacaacccgcagtaagccaggcgcttaagcgacttgaagatcagttaggaaaagtcctagtcaagcggagcggatcgcactttgagattacgcaagcgggaatgctcatccaacggcttgcggaggaaatttacggcaatatttcacaactggcagctgccgatggcgatatcgatgatcgggacatttccggtgtcgcccgcttgttggttgtcagcggaataagttcgaccagctacgatgattttttggcgaaatttcatcgcgcctcaccacgtattgacctgcaaattcaagtgatgagaagcgccgacattgtcaatgctttgcttcaaaagacggcaacggcaggtatcagcccggactatttgctgccaaaaaaaatccagcaacagctatttattccacagaaattttcttttttttgcggaagacatcatcatctgttcgggcggtcgaacctcactgttgctgacctgcgcgacgaaaatatagtgtcggtgtcaggcgaccagatcggtgacaatttttcgaaacttactctatttcgcgagcaaaatggattttccggccgtgtagtcgcttcctcagcaagtatgaccgaaatgcgtcgactcattttttcaggatttggaattggctgtttgcctgaggaaatcgcgcgtagcgacgtcatgcagcaaagactctttcgacttcctccggacgacggcgttgttaatgtcgatttgcatttattatggaatatcgaacgtaactattctcctgcggagtcggtttttctggacagcttgaggtcctatattccgcaacatgccgacaaagcagacgtcgggtaagaataaaccttgacccagatctggaaccaacacgaatatgtcatcaatctgctcagaacgatagctctcactgtggatcaagtcccatgtcttctgcaattcacatctctcactctcgtcgggctgcaattgccagttttgtaggcacaacagttgagttttacgatttctatatctatgctacagccgcagcccttgtgttaggcgaagtcttctttccaaatctcaatcaatttcaaggcacgatggcgtcgtttgccacgtttgcagtcggcttcattgctcgaccattgagtggcatggtattcggtcattttggtgacaggttaggacgcaagaaaatgctggttattacgatgttcctcatgggcattgcaacgactgccattggcctgcttccatcgtatggacaagttggcatctgggctccggctttgctgattttttttcgtattttgcagggagttgcggtcggcggggaatgggggggggccgtgctcatggcatgtgaacacgcgccggccgggcgcaaaacattctttgcgtcgcttcctcaaatgggtagtcctgcgggactgatcctggcgcttctatcgttccgggcagtggcatcaatgtttcacgaaaatctcatttcttggggatggcgcatacctttcctggcgagcttcgtgttattgatcattggggtgttcatccgcataggagtaagtgagtctccggagtttgaatcggttgtgaaggcagagggacttgccaaatttccggtagctgaagtgttacgcactgcatgggtgccaatcttgcttggcgccggcgcgataaccatcagtaccgccggcttctttttcaccaatacctttatgatttcctacgtgacaagctatctcggcatgcaaaagtcgacgattcttgatgtcctcctggtggtgacgatcattcaattatgctcacaacccatttcggcattgcttgcggaacgttacggcaatgctcggttcctgatggccgcagcgttgctgtcaatgttggcaccctacccaatgttcttgctggtcaatacgcaagacccggtcgtcatgatcgtcggcatttcattggctgtgatccctttgtctgctgtctatgctgtcattgccggctttatgatcgcagtgttcccaacgcatctgcgttactcggggatttcgatcgcatatcagttctgctgcgctgtggcaggagggacgacccctttaattggtgcgctaatcgccggcaactataagggggagtggttacctctcgcgctgtatttcagcttgctagccagcgtatcatttgtttgtatatggagccttgcacgttatcaacgtcaacaactattcacgctaaacgtggcagtaggaaaatcgtatgggatgagttagtgcatcgccgattttgaagaaaacgcatgagtgttcgctggcgaagtatcgtgaaaaattttcgcaacagattactgatagcaagccattgttacgaggatgacggtctggcataaaatccgacactgtccatactggttcgtcaaaatgcgtgacagtgtcgatagtctaacgccaagcaacccaagcccatgcgcgacaggcacgaaggcctccactgcgatgtcctgcgaggcagtagtcatccagtctgcgattccaagttcttggttccgtctttagtaatatgcgggccatcttcctgccgtgcatcagacccaagctggagacgtacaattgggccagttggaattgggattgtcatttggctcaggcgtttgaaacgagtcagcccgatcacaacaagtacagcagcggatgaaatcaacccggataggagtgacagcaattcgacacgaccggcaaagaatcctatccatcccgcaaagtataaaccgagggaagtgacaaaaccggcgcccatgataaattgtgcggcttgcggtagcattacgatgcctctctgcaaggcgataaaaagaacggcaatgctggcgatgtacacgatattgactgccaccatggtgtcaataatggcctgcccgcgagcggcaacaatagcgccggcaagcgtgatgataagcatcaaaagggaatgtggcacttgccgttttgtcgaaatggttgacgcaagggctgcagtcattgcacggatgatcgcagcgccggatccaagcgcagcagacagaagaaccaataacaaaccgattgatacgcctgttccggcacgccctgtgaattgcatgagcaaaaatggtatgacctgcttggcatcagcgagtccattcagcatgccagcctggcgtgcggctaacactgctgctgcgggaagaaaacctgttaggaacagaaatcctgccgcaagcaggcaaccgaggatcgcatcaatcgggcgccgtgccgcaataacgaattgctggtaatccgcacctgtaataaccagaaagacgatggcgaccatcgttacgaacagttcatatggccgggccaaatggacgtcttgcataaaaagcggtaccgcttccacgtaaagatgtaggccatcaacgttactcagcgcatacaacagcgcgaggttactggcaaccaagcagagagaaaacaatttcgaagcaagacgtaaattaagcgaacaagcagcaagaagcaacgtgagagaaagcgcataactcagccctgccggcaaaccggccagcttaagaatcgcaacgctgccatgtatctgggctgccaatacaccagccatccagatgagcgacagaagcgcaacactatttttcatttttgcgccataagcccgtccgaacacatcccatatctgcatgccagaagaccaaagtcgtcgagaaaatagcgcgagaaaaaccatgccggtggcggtcgccattgcgtagatgctgccagccatgccgagtctgagcgccagttcccctgatccgaacaaaaagccgatgccatagcttgcggaaacgagcagtgcggcgatatggaggaagttcaaattgcgcgacatcatatatttcttccttatccgttactctggataacagcggcttttccgtaaaaatatgcactgcagcgcacacttcgctccatatagtgagggagtttgatcaacatttcatgtgtttgatgaagccggtaatagggaacggctggcatcaggtggtgcgtgaggtggtacccattatttcgaggatgcacgatccaacgccacggactcaacgttgagacatctcgtgtgaatgagaaaattcctcctggctcaaggccaaaatggtcacacatctctcggaatgtcgttaccagatggaacatactcgctttcgcgatcatccaaagggcaaagaaggttagtgcaaattgaatattcatcacgataacagccatcgcgagaatcgagagccaccagaaataaatataggcggatcgcttgagattcaggtctggtataccgagatgtcccatgacagagccgcgccagcatccgaacgagaaaatatattttcgataagtcaaccaccacgacgattgctctcccgtgattgaagtaatgtaatcaggatcgcgcaatggatcgccaagaaaggcgtgatgtcgtgagtgcaatttgcggtaaatcgccaggctattgaaaagcgctggggccaagagaaatgttgcaatcagatcatttgccttgtccgaattgaacagattacgatggccagcgtcgtgtaggaggttgcccatcgcgcgctgtctatttgctacgacaattatcgctactggtactgcatagcttcccgcgcgccagacaaaataggtcatcgctagaatcgtcagccaatcaagtgcgatgtccagcaatacgcgcatttcgttttttcgctgtaactcaccataggctaaatcgctatctttcagtgccctgtgcaagtcaatttggtccgcgccaataccacatccgtgattcatcattaaatagcttctcggacttctgtttcagttaagggagtgaactcttgcaatagatttggattggccccttgaatcacaggatacgtgaacacctctaaaataagagacagccttgtgactagttttaagcctatgcaaacattgaacaattcgaagtattgacggaatctgaaaggcatcagcgcatccaccgcaggagaaactggtaaacgtccaggaaacattcgaagccgggaaatccatctctccggtggcccgccaatgtggcgtggatgcaaaccaggttttttgctggcgcaagcagtaccaggaaggtagtctgattgcagcctttcccaagccttgaaagaaatcaaggagctgcagcgtattctgggcaagaagactagggagaacgaaattctgcgtgaagtgcggtcgctcaaaaaactggattgggcactcgccattagtgccgcgagacgccgatgaaaaaggtctgtgacgttctccgtgtagagcgctctgcggcagtagtacggctgcacctgtccgggcaaagtaacggcatggctgaatcgttcgtgaagcctatgaggcatgattacatcgcctttttgaataaaccggatctgcctacggcgctcaagcatctggccgcaggatttgagcattacaatgagcgccacctgcacagtgccttgaaaataccgttcttgtatggagttcaggcaccggacgacatcgctaacttaaaggtgtcatggtgttctgagtgacaggggcaaatccaagattcacttattaatctagagcgaggacaagagatgatacgtcgacgctcagcacacatctttagctatgtagaaaggaaaactcaggatctgccttgcgcgcacacaactgccgcatgacggaggcaaacgcgtcaatgtgaatgtaacaaggaattgcaggaaatgtgtaatggcctcgaccttcgctggaccacacgatcctggcaacaagatggaagtagccatacggggttccaaataccctccagcccattgatatacttggaaaccgctacatcgcaaacctattgttccctgtgcttcccgccataagaacgcatcgattatcaaggcatttcgaggcatgatcaaataattgattgttatacggcgtataattttccctaatagtgttctaccccgttttattgtgattacgccgaaattatgccaaagtggctgcggtactgcatgaaataaggaatg

SEQ ID NO: 2 - protein, 265 aa, Collimonas fungivorans
  MISC_FEATURE (1)..(265): ORF1 product=“putative hydrolase”
VSTPAEQLAPTPESVAKAFLTPRTYERAPDTAGFQGASRVETNGPTGKLVSWQAGRGPIVLLVHGWEGRSSDMASFTPPLLAAGYRVVLVDLPAHGESEGTTSSIPACAAALLKLQDLIGPVYAAIAHSVGCALTVEAVRHGLQVERLVLIAPPARCFDYAVGFGVQAGLDRHQVAAMIALLQEQGVDVADIDTPKAAANLKQPVLILHSNDDRVVPVSLGVEIANAWQGAQLLRLDGLGHRRILQAPEVIEAARSFLMARTAVA

SEQ ID NO: 3 - protein, 39 aa, Collimonas fungivorans
  MISC_FEATURE (1)..(39): ORF2 product=“hypothetical  protein”
LDNDAGFIHLAVGQASIAGRQILDVLLQKKETQAYGPSL

SEQ ID NO: 4 - protein, 61 aa, Collimonas fungivorans
  MISC_FEATURE (1)..(61): ORF3 product=“rubredoxin-type  Fe(Cys)4 protein”
MTETETEFKTLMCLVCGWIYSEKDGAPSEGLAAGTRWDDIPDTWTCPECGVKKEDFTMVEF

SEQ ID NO: 5 - protein, 317 aa, Collimonas fungivorans
  MISC_FEATURE (1)..(317): ORF4 product=“delta 12 desaturase”
MTDITSPSPPLSQQPQQPAAAPALGIGASLAKAGYQLGGMLAPALAGRIAADAFGRSRSKGGRTLFRMPLGAQTFEIAGNDDVRHGYLWKNHGPTVLLVHGWGSDSSSMIGFVKPLLALGFQVASFDAPAHGESAGNKTTMTRFVKAVGAAIKSLDNVQVIVGHSLGSIASVAAIAHADARHAEAVKRLVLIAAPVSLSTVLERWSTNQHQQLPPAVIGKIYERLQVQNGVPVSHWDISVLGAAMEVPVLVVHDEHDPVVPLSEAQRLMRSLKNVRLEQTSGLGHSRILSATPVKELITRFIGPSHLTPTSLEPSND

SEQ ID NO: 6 - protein, 366 aa, Collimonas fungivorans
  MISC_FEATURE (1)..(366): ORF5 product=“fatty acid desaturase”
MPSVNSYPSSPGPEFEAFLDKSKTLEGARLAEAIPKSYFEPQAWRGLAGFFISYALYIGAIVGIAYAPHWGFYIPLYIIAGLGGWGLHCIAHDCGHNSFSRNKKLNIVIGHLSLLPLLYPFYSWKHVHNLHHFHTNNLELDTDWRPIPRDMYKRMSFGQRFVYMGTRTWLFWAGTINYWIVSGFRPGFFPKKDMRTDVKRSIAFVVLASIVYFSALIYFTGITGFFLLFFAPWIATHTWFSVTTLMHHTSADIPFLTSEHWTPNASRMLVTTDYRYPKWLLFLTHNISIHTAHHVAPVVPFYNLQKAQVALKQAYPGMIREKDFSFGDLLWVIRNCHFYDLDSGYYKDSAEQRAEVNGGANSVANT

SEQ ID NO: 7 - protein, 110 aa, Collimonas fungivorans
  MISC_FEATURE (1)..(110): ORF6 product=“phosphopantetheine-binding”
MSQENKSGGLFGFFRKGGEEVESNVPLSEEVIRQWLVKRLAKQVKVDPSAIDTAKKFELYGLDSIVAVRVSGDLEKLVEQRLSPALLFEHPSIDELSAHLATELGLNAIA

SEQ ID NO: 8 - protein, 322 aa, Collimonas fungivorans
  MISC_FEATURE (1)..(322): ORF7 product=“ delta-9 acyl-phospholipid desaturase”
MTTQTNVLPPKPASAAGPALRPGVAPLTGAGATIKQITALVVMLVPLAGFALALQRALEGRMSTTDLVLFGVFYFLHMGGITMGFHRYLAHKTFNTSPFFEGLLLICGSMAAQGPIMFWVTTHRRHHTYSDQHGDPHSPNLHGPGLLGRLRGIWYAHMPWMLAKDVSGWNFFAPDILANRKLFFYHRTYGLWIVLGLALPAAIGGAVGGSWDAAFSGFLFGGLARIFLANQAAWCVGSVCHMIGGKPFKTDDNSANNWTVAILTFGEGLQNNHHAFPGSYRHGVKWWEPDLSGWLLAGFGKIGVVWNLRSPDEKAVAKMRAR

SEQ ID NO: 9 - protein, 315 aa, Collimonas fungivorans
  MISC_FEATURE (1)..(315): ORF8 product=“stearoyl-CoA  9-desaturase”
VIDTKNPAAAAPGEKVQALRDGDSKARRLAYLTVLTPAVGFVAALWYSIQFGFKQQDMILFGVMYFLTSFGVEGGLHRFFSHRAFKAGPVVTAVIGILGCMAAQGPILFWAATHRMHHVFTDQDGDPHSPRVLSPGLRGRIKALWHGHVGWLFTVKRSNWSTYVPDLFGSKLVLFVNQHYLIWVFLGLALPAAAGAALSGVEGAVGGLLWGGLARIFLLDQVTWAVNSIGHTFGKRPNQTRDTSGNIGWLALVSAGGGWHNNHHANPALAHNDFHFWQIDTTAWVIRLLGLLGLAWDIRQRQSADPLDQKLFNND

SEQ ID NO: 10 - protein, 617 aa, Collimonas fungivorans
  MISC_FEATURE (1)..(617): ORF9 product=“AMP-dependent  synthetase and ligase”
MTSSLSMSEATVLVPEQAAQDIDMDAILRRRAAATPDEVALRFLKNGEDDIAELTYRQLDLRAAGLARQLKQQLTPGARVLLVLEPGLHYVTALFAIFKAGATAVPSFPPVGSRAVARFASICNDCNAQLVIAEGNLQAAVDRLNASLSAEGNVPEWLFVGSDFFEAQDTQPLPTDENDEAGSSEQFPALLQYTSGSTGNPKGVVLSAANLISNSRVLDIRLGSAEKHIGFTWLPPYHDMGLMGALLLSVYSGFTLVVMTPAHFVQRPLRWLKGLSTHQVTTSVAPNFALDLCVDTITDEEIAELDLSRLKMLFCGAEPVRQATLDRFSEKFKAAGFSSAAFVPCYGLAEATLFISGKANRDATPKTLLLDHEALACGQAKPATADAPATAVISCGTVASGHVMAIVDPDTRQPLPAGAVGELWFKGASVAQAYLNQPEASTETFHAFIADDRQDGPYLRTGDLGFMLDDELYITGRIKDVIIFAGRNLYPQDIEAAAQASHPAIRTNGVAAFSVTRDDTEQLVVVAEILRSAKLSGEDLENVEDAIAAAITRSHGVAPHTVHLAPVSTIPLTTSGKVRRSACRDAFNAGSLAYAKARPTPSATTTETDSTTLFQQG

SEQ ID NO: 11 - protein, 472 aa, Collimonas fungivorans
  MISC_FEATURE (1)..(472): ORF10 product=“major facilitator superfamily MFS 1”
MNVPLAPALQAQSSSISPWPVFWIASVAVFLVSIDVTLLYAAFPALQRAFPEAGSAHLSWVLTGYTLVFAALLVPAGRFADLRGRKRTFLLGLAMFLGGSLGCGLARDVATLIAMRVAQGAGAALLFPASLSILLAAFPASKRAVAVGLWGAVSGVAGALGPSLGSFLVDRFGWPWAFFLNLPLGAITLWLGWRILDESRDPERGAPIDVIGVVLLILGVGAITFGLVQSEAVGWMSPMVILAIAGGLATLGAFVVWARTVRAPAIDLSLFQDRTYCYINLASLFFAIGFAMMFFQTFLFTTGVWSYSLTRAGLAGSLGPLLVVPTAIVCGRFAARAGHKLLLVTGSLISTAASIWFALVPGINPDYLHTWLPGALLTGLGVGMVMPSLSAAAVAHLPPARFGVGSAVNQAVRQMGSVLGVALTVAITGHATLNLAAFHTLCYLQIALGFATAILCVPVDTRSRAPAPATAH

SEQ ID NO: 12 - protein, 281 aa, Collimonas fungivorans
  MISC_FEATURE (1)..(281): ORF11 product=“hypothetical  protein”
MNATLTEKETPMSRQRHAGLYVDELLRELETSAQDEVSLRHPMTFGEVVGHGRVEQLAAAVESIFSEVEPDHTWSGDDIGGREWHGQFQDYRIRGVTVARRFGQRVQVDIFLASFPAPVRQALHDACRDLIDADAWLLPPGTDAALPPIPTDEVLDAKLPFGLAANAFLTSPVACKPIRGAAYVERLCGHSIVVYGGRANGPRLTAGATTLSLWTGSVAGLPLEVANVIHWEDAQHVKVMAMAMRPWPVVALFQARMQARTLSFLDASYFESGDGATSPPA

SEQ ID NO: 13 - protein, 344 aa, Collimonas fungivorans
  MISC_FEATURE (1)..(344): ORF12 product=“Rieske  (2Fe-2S) domain protein”
MFIQNAWYAAALSGEIKSEPFARTILNEKIVMYRTSANQVVALEDRCAHRQVPLSRGRLIGDELQCWYHGLRYDCSGECVNIPSQSTIPKGARVRSFPAVEKHGFVWLWPGDPDQADESAIPNHWVCSAPELAGTMSYCTIACNYVFGIDNILDISHAAFVHAKTLGSLDIVETPPEIVINDDEVRVRRYMRREKTPPLYSRILQLDYIDRFQEVLYWPVGNTRVETRAHACDDADGKVFHVYTTTIFTPATDTTSHVFVGMHRDFDIDNHGFTEFTAKEVFSTVMEDKDVAESLQANWSPTAPMIDIHLDRPAFAARRILERMGANASIRTGHGTPLPQFEEV

SEQ ID NO: 14 - protein, 360 aa, Collimonas fungivorans
  MISC_FEATURE (1)..(360): ORF13 product=“fatty acid desaturase”
MTIEASLEADTAFARLKLPEKFHRTSHFATALYLAHALAFFLVPAMVAFAIVDIPASTPVRIGLALVLGIIGGHGMHLLTFVGHEGMHTNLHRNKYISAALAVLFSSLVPFFFIVGFSMTHWKHHRFTDQDIDPDAQIYSQYNNFVSRFFLARSGTVRVYTKNALRMALGLPWPENTKLPFSEREMRWITRINIALGLCGVTVYALIWHRSLWLGFTVMLVPYFGLYVMSAMRAYLEHTGTAPGRYHDARSYTSPIYTALFFGSNFHLEHHLYPAVPCYRLPALHGHLASQGLIKATGAQVERSFFGALRYTTGRFPYPHVELPSKADDFLERIADGRFDREADSAGTRAGEAMVAVVRE

SEQ ID NO: 15 - protein, 461 aa, Collimonas fungivorans
  MISC_FEATURE (1)..(461): ORF14 product=“monooxygenase,  FAD-binding ”
VLDTLIVGARCAGSTLAIHLARAGKSVLAIDANNLPSDQPLSTHWISPHGMALLDELGLGDKVRRFAPPVPSMMYGADEEIGRVVYPAGGRCSCPRRMDLDALLADEARAAGAEIRPRTKLVELLRDGERVTGAVVEHAGTREEIRARIVVGADGQHSTTAQLVSAEEYHGYDCPRAMYWAYWPRPTWFDDDPRHLGGAMNCSFGDDVVFIFPTNRDLLLIGVAFPRTQLPEWKGRALQKLQETLQRNPYTAPLTAGEPKSKVLGALNLRFFFRQAAGPGWALVGDAGLFMDPSPGFGITDALRDARSLGRAIIEGGDQALVRYWRQRDATSIDLFEYSRSRGALTNNNPLSRMLFGKLAADPTLQARLLSAQNRQCSPFAVFNPSDIKKWADEAVERGEVGVIQPLIEMSRRGEEIRSEMALRQRLAEQANATPVRAPEFDQSNLLASSADHALGVSEPV

SEQ ID NO: 16 - protein, 430 aa, Collimonas fungivorans
  MISC_FEATURE (1)..(430): ORF15 product=“beta-ketoacyl  synthase”
MMRRVVVTGLGIVSPLGCGTELVWQRLIDGRSGLRVLGDEIVGDLPVKVGGTVQDRTADPEGGFAPEISIPSKELKKMDRFIQMAMVAAEEALADAGWAPQTDHQCERTATIIASGIGGFLGLANAVRIGESPGVRRLSPFTIPSFLANLAAGQVSIKHRFRGPLGCPVTACAASVQAIGDAMRLIRAGEADIVLAGGAEAAFDKVSLGGFSAARALSTGFNEQPVRASRPFDRDRDGFVMGEGAAMLVVEALDHAIARGAIPIAEIIGYGTTADAYHMTAGPDDGNGAMRAMRLALAMAGIAPERVDYVNAHATSTPVGDAGEIAALKTIFGTSTGPAISSTKSATGHLLGAAGAIEAAFSVLALRDGVLPGTLNLEHLDDSAIGLDLIGPSARRAPIEIALSNGFGFGGVNASVLFQRFSAERKENVR

SEQ ID NO: 17 - protein, 57 aa, Collimonas fungivorans
  MISC_FEATURE (1)..(57): ORF16 product=“hypothetical  protein”
LGEKTFNQITIAILAPVKFSLFFPILSLRDNGRGIFYCNTSKLFATSKICLAKSGWL

SEQ ID NO: 18 - protein, 326 aa, Collimonas fungivorans
  MISC_FEATURE (1)..(326): ORF17 product=“transcriptional  regulator, LysR family”
MMNMADPIKPRVKSSLLNDRLDWNLLRTFMVIAKERSISRAAGKLHLTQPAVSQALKRLEDQLGKVLVKRSGSHFEITQAGMLIQRLAEEIYGNISQLAAADGDIDDRDISGVARLLVVSGISSTSYDDFLAKFHRASPRIDLQIQVMRSADIVNALLQKTATAGISPDYLLPKKIQQQLFIPQKFSFFCGRHHHLFGRSNLTVADLRDENIVSVSGDQIGDNFSKLTLFREQNGFSGRVVASSASMTEMRRLIFSGFGIGCLPEEIARSDVMQQRLFRLPPDDGVVNVDLHLLWNIERNYSPAESVFLDSLRSYIPQHADKADVG

SEQ ID NO: 19 - protein, 440 aa, Collimonas fungivorans
  MISC_FEATURE (1)..(440): ORF18 product=“major facilitator family transporter”
MSSAIHISHSRRAAIASFVGTTVEFYDFYIYATAAALVLGEVFFPNLNQFQGTMASFATFAVGFIARPLSGMVFGHFGDRLGRKKMLVITMFLMGIATTAIGLLPSYGQVGIWAPALLIFFRILQGVAVGGEWGGAVLMACEHAPAGRKTFFASLPQMGSPAGLILALLSFRAVASMFHENLISWGWRIPFLASFVLLIIGVFIRIGVSESPEFESVVKAEGLAKFPVAEVLRTAWVPILLGAGAITISTAGFFFTNTFMISYVTSYLGMQKSTILDVLLVVTIIQLCSQPISALLAERYGNARFLMAAALLSMLAPYPMFLLVNTQDPVVMIVGISLAVIPLSAVYAVIAGFMIAVFPTHLRYSGISIAYQFCCAVAGGTTPLIGALIAGNYKGEWLPLALYFSLLASVSFVCIWSLARYQRQQLFTLNVAVGKSYGMS

SEQ ID NO: 20 - protein, 457 aa, Collimonas fungivorans
  MISC_FEATURE (1)..(457): ORF19 product=“conserved hypothetical protein”
MMSRNLNFLHIAALLVSASYGIGFLFGSGELALRLGMAGSIYAMATATGMVFLALFSRRLWSSGMQIWDVFGRAYGAKMKNSVALLSLIWMAGVLAAQIHGSVAILKLAGLPAGLSYALSLTLLLAACSLNLRLASKLFSLCLVASNLALLYALSNVDGLHLYVEAVPLFMQDVHLARPYELFVTMVAIVFLVITGADYQQFVIAARRPIDAILGCLLAAGFLFLTGFLPAAAVLAARQAGMLNGLADAKQVIPFLLMQFTGRAGTGVSIGLLLVLLSAALGSGAAIIRAMTAALASTISTKRQVPHSLLMLIITLAGAIVAARGQAIIDTMVAVNIVYIASIAVLFIALQRGIVMLPQAAQFIMGAGFVTSLGLYFAGWIGFFAGRVELLSLLSGLISSAAVLVVIGLTRFKRLSQMTIPIPTGPIVRLQLGSDARQEDGPHITKDGTKNLESQTG

SEQ ID NO: 21 - protein, 310 aa, Collimonas fungivorans
  MISC_FEATURE (1)..(310): ORF20 product=“fatty acid desaturase”
MMNHGCGIGADQIDLHRALKDSDLAYGELQRKNEMRVLLDIALDWLTILAMTYFVWRAGSYAVPVAIIVVANRQRAMGNLLHDAGHRNLFNSDKANDLIATFLLAPALFNSLAIYRKLHSRHHAFLGDPLRDPDYITSITGEQSSWWLTYRKYIFSFGCWRGSVMGHLGIPDLNLKRSAYIYFWWLSILAMAVIVMNIQFALTFFALWMIAKASMFHLVTTFREMCDHFGLEPGGIFSFTRDVSTLSPWRWIVHPRNNGYHLTHHLMPAVPYYRLHQTHEMLIKLPHYMERSVRCSAYFYGKAAVIQSNG